(19) United States
(12) Patent Application Publication
(10) Pub. No.: US 2007/0042405 A1
(43) Pub. Date: Feb. 22, 2007

(54) Enhanced diagnostic multimarker serological profiling

(75) Inventor: Anna Lokshin, Pittsburgh, PA (US)

Correspondence Address: THE WEBB LAW FIRM, P.C., 700 KOPPERS BUILDING, 436 SEVENTH AVENUE, PITTSBURGH, PA, 15219, US

(73) Assignee: University of Pittsburgh -of the Commonwealth System of Higher Education, Pittsburgh, PA (US)

(21) Appl. No.: 11/477,143

(22) Filed: Jun. 28, 2006

Related U.S. Application Data
(63) Continuation-in-part of application No. 11/104,874, filed on Apr. 13, 2005.
Application No. 11/104,874 is a continuation-in-part of application No. 10/918,727, filed on Aug. 13, 2004.
(60) Provisional application No. 60/495,547, filed on Aug. 15, 2003.

(51) Int. Cl. C12Q 1/68; G01N 33/574; G06F 19/00

(52) U.S. Cl. 435/6; 702/19; 702/20; 435/7.23

(57) ABSTRACT

The present invention is related to methods of early diagnosis of ovarian cancer in a patient by determining serum levels of blood markers using a novel LabMAP™ technology (Luminex Corp., Austin, Tex.), which allows for simultaneous measurement of the blood markers in serum. The panel of blood markers offers extremely high predictive power for discrimination of ovarian cancer from both healthy control patients and from patients with benign pelvic/ovarian tumors. The methods of the present invention allow for rapid, early diagnosis of ovarian cancer with extremely high sensitivity and specificity to be clinically useful in disease diagnosis.

### CROSS REFERENCE TO RELATED APPLICATIONS

The present application is a continuation-in-part of U.S. Patent Application Ser. No. 11/104,874, filed Apr. 13, 2005, which is a continuation-in-part of U.S. Patent Application Ser. No. 10/918,727, filed Aug. 13, 2004, which claims priority to U.S. Provisional Patent Application No. 60/495,547, filed Aug. 15, 2003, all of which is incorporated herein by reference.

### BACKGROUND OF THE INVENTION

1. Field of the Invention

The present invention is related to methods and reagents for a multifactorial assay for the rapid, early detection of cancer and, more particularly, is related to a multimarker serological diagnostic test for early detection of ovarian cancer.

2. Description of Related Art

Ovarian cancer represents the third most frequent cancer of the female genital tract. The majority of early-stage cancers are asymptomatic, and over three-quarters of the diagnoses are made at a time when the disease has already established regional or distant metastases. Despite aggressive cytoreductive surgery and platinum-based chemotherapy, the 5-year survival for patients with clinically advanced ovarian cancer is only 15 to 20%, although the cure rate for stage I disease is usually greater than 90% (Holschneider, C. H. and J. S. Berek, Semin. Surg. Oncol., 19(1):3-10, 2000). These statistics provide the primary rationale to improve ovarian cancer screening and early identification.

Epithelial ovarian cancer is so deadly in part because of lack of effective early detection methods. If detected early, survival is dramatically increased. Current research now is focusing on developing improved ways of evaluating women, particularly those at high risk to develop ovarian cancer. As yet, however, a premalignant lesion has not been identified. Although alterations of several genes, such as c-erb-B2, c-myc, and p53, have been identified in a significant fraction of ovarian cancers, none of these mutations is diagnostic of malignancy or predictive of tumor behavior over time (Veikkola, T. et al., Cancer Res., 60(2):203-12, 2000,; Berek, J. S. et al., Am. J. Obstet. Gynec., 164(4):1038-42; discussion 1042-3, 1991; Cooper, B. C., et al., Clin. Cancer Res., 8(10):3193-7, 2002,; and Di Blasio, A. M. et al., J. Steroid Biochem. Mol. Biol., 53(1-6):375-9, 1995). Instead, high-risk women must rely on genetic counseling and testing, as well as measurement of serum CA-125 levels and transvaginal ultrasound (Oehler, M. K. et al., Anticancer Res., 20(6D):5109-12, 2000,; Santin, A. D. et al., Eur. J. Gynaecol. Oncol., 20(3):177-81, 1999; and Senger, D. R. et al., Science, 219(4587):983-5, 1983). CA-125, however, is neither sensitive nor specific for detecting early stage disease. Current recommendations do not favor it for general screening. It is only thought to be robust in monitoring the response or progression of the disease, but not as a diagnostic or prognostic marker (Gadducci, A. et al., Anticancer Res., 19(2B):1401-5, 1999).

Screening using transvaginal ultrasound, Doppler and morphological indices has shown some encouraging results but, used alone, it currently lacks the specificity required of a screening test for the general population (Karayiannakis, A. J. et al., Surgery, 131(5):548-55, 2002,; Lee, J. K. et al., Int. J. Oncol., 17(1):149-52, 2000). Combinational multimodal screening using tumor markers and ultrasound yields higher sensitivity and specificity. This combination approach also is the most cost-effective potential screening strategy (Karayiannakis et al., 2002; Lee et al., Int. J. Oncol., 2000). However, it, too, is of questionable effectiveness in the general population. Thus, there is a critical need to develop additional markers for early detection of disease.

Recently, a novel technology named Surface-Enhanced Laser Desorption/Ionization Time-of-Flight Mass Spectrometry (SELDI-TOF-MS) that combines solid phase protein chromatography and mass spectrometry (reviewed in Issaq, H. J. et al., Biochem Biophys Res Commun, 292(3):587-92, 2002) has been utilized as a novel approach to biomarker discovery in ovarian cancer. In a recently published landmark study of ovarian cancer patients, the new technique has been utilized for protein profiling of ovarian cancer progression (Petricoin, E. F. et al., Lancet, 359(9306):572-7, 2002). This approach allowed for the discrimination of serum protein profiles with a positive predictive value of 94% as compared with 34% for CA-125. However, as high as this value is, due to the low incidence of ovarian cancer in the population likely to be screened, the positive predictive value must be almost 100% to avoid generating a high number of false positives. Thus, additional markers are necessary to provide the required high level of specificity and positivity that are required to utilize this approach for the effective general population screening for ovarian cancer. Additionally, this approach is very expensive and could only be applied to high-risk populations.

It is well known that ovarian cancer cells produce various angiogenic factors and stimulate secretion of various cytokines, which potentially can be used as biomarkers. However, each single factor has been shown to only weakly be associated with early stage disease. It was hypothesized that evaluation of a panel of angiogenic factors and cytokines in the serum of each individual patient would provide sufficient specificity and sensitivity for diagnosis of early stages of ovarian cancer. All previous testing of serum markers of cancer patients had been performed using ELISA, which is very expensive and requires a separate kit for each individual cytokine.

There exists a critical need, therefore, to provide a relatively non-invasive screening test having high sensitivity and specificity in order to facilitate early diagnosis of ovarian cancer.

### SUMMARY OF THE INVENTION

The present invention fulfills this need by providing methods of early diagnosis of ovarian cancer in a patient by determining serum levels of blood markers using a novel LabMAP™ technology (Luminex Corp., Austin, Tex.), which allows for simultaneous measurement of the blood markers in serum. The panel of blood markers offers extremely high predictive power for discrimination of ovarian cancer from both healthy control patients and from patients with benign pelvic/ovarian tumors. The methods of the present invention allow for rapid, early diagnosis of ovarian cancer with extremely high sensitivity and specificity to be clinically useful in disease diagnosis.

In particular, the present invention provides a method for early diagnosis of the presence of ovarian cancer in a patient comprising determining levels of markers in a blood marker panel comprising two or more of EGF (epidermal growth factor), G-CSF (granulocyte colony stimulating factor), IL-6 (Interleukin 6, with “IL”, as used herein, referring to “Interleukin”), IL-8, CA-125 (Cancer Antigen 125), VEGF (vascular endothelial growth factor), MCP-1 (monocyte chemoattractant protein-1), anti-IL6, anti-IL8, anti-CA-125, anti-c-myc, anti-p53, anti-CEA, anti-CA 15-3, anti-MUC-1, anti-survivin, anti-bHCG, anti-osteopontin, anti-PDGF, anti-Her2/neu, anti-Akt1, anti-cytokeratin 19, cytokeratin 19, EGFR, CEA, kallikrein-8, M-CSF, FasL, ErbB2 and Her2/neu in a sample of the patient's blood, where the presence of two or more of the following conditions indicates the presence of ovarian cancer in the patient: EGF_{LO}, G-CSF_{HI}, IL-6_{HI}, IL-8_{HI}, VEGF_{HI}, MCP-1_{LO}, anti-IL-6_{HI}, anti-IL-8_{HI}, anti-CA-125_{HI}, anti-c-myc_{HI}, anti-p⁵³_{HI}, anti-CEA_{HI}, anti-CA 15-3_{HI}, anti-MUC-1_{HI}, anti-survivin_{HI}, anti-bHCG_{HI}, anti-osteopontin_{HI}, anti-Her2/neu_{HI}, anti-Akt1_{HI}, anti-cytokeratin 19_{HI}, anti-PDGF_{HI}, CA-125_{HI}, cytokeratin 19_{HI}, EGFR_{LO}, Her2/neu_{LO}, CEA_{HI}, FasL_{HI}, kallikrein-8_{LO}, ErbB2_{LO} and M-CSF_{LO}. Exemplary panels include, without limitation: CA-125, cytokeratin-19, FasL, M-CSF; cytokeratin-19, CEA, Fas, EGFR, kallikrein-8; CEA, Fas, M-CSF, EGFR, CA-125; cytokeratin 19, kallikrein 8, CEA, CA 125, M-CSF; kallikrein-8, EGFR, CA-125; cytokeratin-19, CEA, CA-125, M-CSF, EGFR; cytokeratin-19, kallikrein-8, CA-125, M-CSF, FasL; cytokeratin-19, kallikrein-8, CEA, M-CSF; cytokeratin-19, kallikrein-8, CEA, CA-125; CA 125, cytokeratin 19, ErbB2; EGF, G-CSF, IL-6, IL-8, VEGF and MCP-1; anti-CA 15-3, anti-IL-8, anti-survivin, anti-p53 and anti c-myc; anti-CA 15-3, anti-IL-8, anti-survivin, anti-p53, anti c-myc, anti-CEA, anti-IL-6, anti-EGF; and anti-bHCG.

The present invention also provides a method for early diagnosis of the presence of ovarian cancer in a patient, comprised of measuring serum levels of a panel of eight blood markers comprised of CA-125, CA-19-9, EGFR, eotaxin, G-CSF, IL-2R (optionally substituted with prolactin), sV-CAM and MIF, in which a significant increase in the serum concentrations of CA-125, CA-19-9, IL-2R, MIF and prolactin in the patient compared to healthy matched controls or patients with benign ovarian tumors, and a significant decrease in the serum levels of EGFR, eotaxin and sV-CAM, in the patient compared to healthy matched controls or patients with benign ovarian tumors, indicates a diagnosis of ovarian cancer in the patient.

The present invention further provides a method for early diagnosis of ovarian cancer in a patient comprising determining the levels of at least four markers in the blood of a patient, where at least two different markers are selected from CA-125, prolactin, HE4 (human epididymis protein 4), sV-CAM and TSH; and where a third marker and a fourth marker are selected from CA-125, prolactin, HE4, sV-CAM, TSH, cytokeratin, sI-CAM, IGFBP-1, eotaxin and FSH, where each of the third marker and fourth marker selected from the above listed markers is different from each other and different from either of the first and second markers, and where dysregulation of at least the four markers indicates high specificity and sensitivity for a diagnosis of ovarian cancer.

The present invention still further provides a method for early diagnosis of ovarian cancer in a patient comprising determining the levels of at least eight markers in the blood of a patient, wherein at least four different markers are selected from the group consisting of CA-125, prolactin, HE4, sV-CAM, and TSH and wherein a fifth marker, a sixth marker, a seventh marker and an eighth marker are selected from the group consisting of CA-125, prolactin, HE4, sV-CAM, TSH, cytokeratin, sI-CAM, IGFBP-1, eotaxin and FSH, and further wherein each of said fifth marker, said sixth marker, said seventh marker and said eighth marker is different from the other and is different from any of said at least four markers, wherein dysregulation of said at least eight markers indicates high specificity and sensitivity for a diagnosis of ovarian cancer.

The present invention also provides a method for early diagnosis of ovarian cancer in a patient comprising determining the levels of markers in a blood marker panel comprising at least two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four or twenty five of fifty-one blood markers comprising CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein 8, leptin, kallikrein 10, MPO, se-selectin, IL-6, TNF-a, ErbB2, prolactin, HE4, sV-CAM, TSH, cytokeratin, sI-CAM, IGFPB-1, AFP, IP-10, MIP-1a, Fas, tPAI 1, CA 15-3, TNF-RI, FAS L, VEGF and NGF, wherein dysregulation of the at least two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four or twenty-five markers indicates high specificity and sensitivity for a diagnosis of ovarian cancer.

The present invention also provides a method for early diagnosis of ovarian cancer in a patient comprising determining the levels of at least four markers in the blood of a patient, wherein at least one marker is selected from the group consisting of HE4 and eotaxin and wherein other markers are selected from the group consisting of CA-1 25, prolactin, HE4, sV-CAM, TSH, cytokeratin, sI-CAM, IGFBP-1 and FSH, and further wherein each of the other markers is different from the other and different from either of the at least one marker, wherein dysregulation of the at least four markers indicates high specificity and sensitivity for a diagnosis of ovarian cancer.

The present invention also provides a method for early diagnosis of ovarian cancer in a patient comprising determining the level of at least one marker selected from TSH, IGFBPI, LH, FSH, sV-CAM, MMP-2, EGFR, ErbB2 and GH in the blood of a patient, wherein dysregulation of the at least one marker indicates high specificity and sensitivity for a diagnosis of ovarian cancer.

The present invention also provides a method for early diagnosis of ovarian cancer in a patient comprising determining the levels of markers in a blood marker panel comprising at least two, three, four or five of eleven blood markers comprising TSH, IGFBPI, LH, FSH, sV-CAM, MMP-2, EGFR, ErbB2, GH, CA 72-4 and CA 19-8, wherein dysregulation of the at least two, three, four or five markers indicates high specificity and sensitivity for a diagnosis of ovarian cancer.

The present invention also provides a method of diagnosing ovarian cancer in a patient, comprising determining the levels of at least one marker from each of the following functional groups: cancer antigens such as CA-125, CEA, CA 72-4, CA 19-9 and CA 15-3; cytokines such as MIF, G-CSF, IL-8, MIP-1b, MCP-1, IL-2R, IL-6, TNF-a, IP-10, MIP-1a and TNFR I; hormones such as FSH, resistin, GH, LH, ACTH, TSH, SMR (soluble mesothelin-related protein), mesothelin (IgY), adiponectin, leptin, kallikrein 8, kallikrein 10, MPO, prolactin, HE4 (and AFP (a-fetoprotein); growth/angiogenic factors such as EGFR, HGF, ErbB2, IGFPB-1, VEGF and NGF; metastasis-related molecules such as MMP-2, MMP-3, PAI-I (active), sE-selectin, sV-CAM, cytokeratin, sI-CAM and tPAI 1; and apoptosis-related molecules such as sFASL, sFAS, Fas and FAS L, wherein dysregulation of the at least one marker from each of the functional groups indicates high specificity and sensitivity for a diagnosis of ovarian cancer.

The present invention also provides a method for early diagnosis of ovarian cancer in a patient comprising determining the levels of markers in a blood marker panel comprising at least two or at least five of EGF, G-CSF, IL-6, IL-8, CA-125, VEGF, MCP-1, cytokeratin 19, EGFR, CEA, kallikrein-8, M-CSF, FAS L, ErbB2 and Her2/neu, wherein dysregulation of the at least two or all five markers indicates high specificity and sensitivity for a diagnosis of ovarian cancer.

The present invention also provides a method for early diagnosis of ovarian cancer in a patient comprising determining the levels of markers in a blood marker panel comprising at least two or at least ten of CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein 8, leptin, kallikrein 10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI 1, CA 15-3, TNF-RI, FAS L, VEGF and NGF, wherein dysregulation of the at least two or at least ten markers compared to a control sample comprised of patients with benign pelvic tumors indicates high specificity and sensitivity for a diagnosis of ovarian cancer.

The present invention also provides an array comprising binding reagent types specific to any two or more of EGF, G-CSF, IL-6, IL-8, CA-125, VEGF, MCP-1, anti-c-myc, anti-p53, anti-CEA, anti-CA 15-3, anti-MUC-1, anti-survivin, anti-bHCG, anti-osteopontin, anti-PDGF, cytokeratin 19, CEA, kallikrein-8, M-CSF, EGFR and Her2/neu, wherein each binding reagent type is attached independently to one or more discrete locations on one or more surfaces of one or more substrates. The substrates may be beads comprising an identifiable marker, wherein each binding reagent type is attached to a bead comprising a different identifiable marker than beads to which a different binding reagent is attached. The identifiable marker may comprise a fluorescent compound or a quantum dot.

The present invention further provides an array comprised of binding reagent types specific to a panel of eight blood markers comprised of CA-125, CA-19-9, EGFR, eotaxin, G-CSF, IL-2R (optionally substituted with prolactin), sV-CAM and MIF, in which each binding reagent type is attached independently to one or more discrete locations on one or more surfaces of one or more substrates. The substrates may be beads comprising an identifiable marker, wherein each binding reagent type is attached to a bead comprising a different identifiable marker than beads to which a different binding reagent is attached. The identifiable marker may comprise a fluorescent compound or a quantum dot.

The present invention still further provides a method of predicting the onset of ovarian cancer in a patient, comprised of determining the change in concentration at two or more time points of CA-125, CA-19-9, EGFR, eotaxin, G-CSF, IL-2R (optionally substituted with prolactin), sV-CAM and MIF in a patient's blood, wherein an increase in the serum levels of CA-125, CA-19-9, IL-2R, MIF and prolactin in the patent's blood between the two time points and a decrease in the serum levels of EGFR, eotaxin and sV-CAM in the patient's blood between the two time points are predictive of the onset of ovarian cancer.

The present invention also provides a method for comparing the serum levels of the markers set forth herein in a blood marker panel with levels of the same markers in one or more control samples by applying a statistical method such as linear regression analysis, classification tree analysis and heuristic naive Bayes analysis.

### BRIEF DESCRIPTION OF THE DRAWINGS

FIG. 1 provides the breakdown of patient groups, age and histologic types of ovarian cancer and benign tumors;

FIG. 2 lists the initial screening panel of luminex analytes;

FIG. 3 provides statistical data of the validation set between ovarian cancer and healthy control groups; and

FIG. 4 provides statistical data of the validation set between ovarian cancer and benign groups.

### DESCRIPTION OF THE PREFERRED EMBODIMENTS

The present invention provides methods of early diagnosis of ovarian cancer in a patient by determining serum levels of blood markers using a novel LabMAP™ technology (Luminex Corp., Austin, Tex.), which allows for simultaneous measurement of the blood markers in serum. The panel of blood markers offers extremely high predictive power for discrimination of ovarian cancer from both healthy control patients and from patients with benign pelvic/ovarian tumors. The methods of the present invention allow for rapid, early diagnosis of ovarian cancer with extremely high specificity and sensitivity to be clinically useful in disease diagnosis.

In an embodiment of the present invention, a method is provided for early diagnosis of the presence of ovarian cancer in a patient comprising determining levels of markers in a blood marker panel comprising two or more of EGF (epidermal growth factor), G-CSF (granulocyte colony stimulating factor), IL-6 (Interleukin 6, with “IL”, as used herein, referring to “Interleukin”), IL-8, CA-125 (Cancer Antigen 125), VEGF (vascular endothelial growth factor), MCP-1 (monocyte chemoattractant protein-1), anti-IL6, anti-IL8, anti-CA-125, anti-c-myc, anti-p53, anti-CEA, anti-CA 15-3, anti-MUC-1, anti-survivin, anti-bHCG, anti-osteopontin, anti-PDGF, anti-Her2/neu, anti-Akt1, anti-cytokeratin 19, cytokeratin 19, EGFR, CEA, kallikrein-8, M-CSF, FasL, ErbB2 and Her2/neu in a sample of the patient's blood, where the presence of two or more of the following conditions indicates the presence of ovarian cancer in the patient: EGF_{LO}, G-CSF_{HI}, IL-6_{HI}, IL-8_{HI}, VEGF_{HI}, MCP-1_{LO}, anti-IL-6_{HI}, anti-IL-8_{HI}, anti-CA-125_{HI}, anti-c-myc_{HI}, anti-p53_{HI}, anti-CEA_{HI}, anti-CA 15-3_{HI}, anti-MUC-1_{HI}, anti-survivin_{HI}, anti-bHCG_{HI}, anti-osteopontinHI, anti-Her2/neu_{HI}, anti-Akt1_{HI}, anti-cytokeratin 19_{HI}, anti-PDGF_{HI}, CA-125_{HI}, cytokeratin 19_{HI}, EGFR_{LO}, Her2/neu_{LO}, CEA_{HI}, FasL_{HI}, kallikrein-8_{LO}, ErbB2_{LO} and M-CSF_{LO}. Exemplary panels include, without limitation: CA-125, cytokeratin-19, FasL, M-CSF; cytokeratin-19, CEA, Fas, EGFR, kallikrein-8; CEA, Fas, M-CSF, EGFR, CA-125; cytokeratin 19, kallikrein 8, CEA, CA 125, M-CSF; kallikrein-8, EGFR, CA-125; cytokeratin-19, CEA, CA-125, M-CSF, EGFR; cytokeratin-19, kallikrein-8, CA-125, M-CSF, FasL; cytokeratin-19, kallikrein-8, CEA, M-CSF; cytokeratin-19, kallikrein-8, CEA, CA-125; CA 125, cytokeratin 19, ErbB2; EGF, G-CSF, IL-6, IL-8, VEGF and MCP-1; anti-CA 15-3, anti-IL-8, anti-survivin, anti-p53 and anti c-myc; anti-CA 15-3, anti-IL-8, anti-survivin, anti-p53, anti c-myc, anti-CEA, anti-IL-6, anti-EGF; and anti-bHCG.

In another embodiment, a method is provided for early diagnosis of the presence of ovarian cancer in a patient, comprised of measuring serum levels of a panel of eight blood markers comprised of CA-125, CA-19-9, EGFR, eotaxin, G-CSF, IL-2R (optionally substituted with prolactin), sV-CAM and MIF, in which a significant increase in the serum concentrations of CA-125, CA-19-9, IL-2R, MIF and prolactin in the patient compared to healthy matched controls or patients with benign ovarian tumors, and a significant decrease in the serum levels of EGFR, eotaxin and sV-CAM, in the patient compared to healthy matched controls or patients with benign ovarian tumors, indicates a diagnosis of ovarian cancer in the patient.

In still another embodiment, a method is provided for early diagnosis of ovarian cancer in a patient comprising determining the levels of at least four markers in the blood of a patient, where at least two different markers are selected from CA-125, prolactin, HE4, sV-CAM and TSH; and where a third marker and a fourth marker are selected from CA-125, prolactin, HE4, sV-CAM, TSH, cytokeratin, sI-CAM, IGFBP-1, eotaxin and FSH, where each of the third marker and fourth marker selected from the above listed markers is different from each other and different from either of the first and second markers, and where dysregulation of at least the four markers indicates high specificity and sensitivity for a diagnosis of ovarian cancer.

In a further embodiment, a method is provided for early diagnosis of ovarian cancer in a patient comprising determining the levels of at least eight markers in the blood of a patient, wherein at least four different markers are selected from CA-125, prolactin, HE4, sV-CAM and TSH and wherein a fifth marker, a sixth marker, a seventh marker and an eighth marker are selected from CA-125, prolactin, HE4, sV-CAM, TSH, cytokeratin, sI-CAM, IGFBP-1, eotaxin and FSH, and further wherein each of said fifth marker, said sixth marker, said seventh marker and said eighth marker is different from the other and is different from any of said at least four markers, wherein dysregulation of said at least eight markers indicates high specificity and sensitivity for a diagnosis of ovarian cancer.

In still a further embodiment, a method is provided for early diagnosis of ovarian cancer in a patient comprising determining the levels of markers in a blood marker panel comprising at least two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four or twenty-five of fifty-one blood markers comprising CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein 8, leptin, kallikrein 10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI 1, CA 15-3, TNF-RI, FAS L, VEGF and NGF, wherein dysregulation of the at least two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four or twenty-five markers indicates high specificity and sensitivity for a diagnosis of ovarian cancer.

In still another embodiment, a method is provided for early diagnosis of ovarian cancer in a patient comprising determining the levels of at least four markers in the blood of a patient, wherein at least one marker is selected from the group consisting of HE4 and eotaxin and wherein other markers are selected from the group consisting of CA-125, prolactin, HE4, sV-CAM, TSH, cytokeratin, sI-CAM, IGFBP-1 and FSH, and further wherein each of the other markers is different from the other and different from either of the at least one marker, wherein dysregulation of the at least four markers indicates high specificity and sensitivity for a diagnosis of ovarian cancer.

In still a further embodiment, a method is provided for early diagnosis of ovarian cancer in a patient comprising determining the level of at least one marker selected from TSH, IGFBPI, LH, FSH, sV-CAM, MMP-2, EGFR, ErbB2 and GH in the blood of a patient, wherein dysregulation of the at least one marker indicates high specificity and sensitivity for a diagnosis of ovarian cancer.

In still another embodiment, a method is provided for early diagnosis of ovarian cancer in a patient comprising determining the levels of markers in a blood marker panel comprising at least two, three, four or five of eleven blood markers comprising TSH, IGFBPI, LH, FSH, sV-CAM, MMP-2, EGFR, ErbB2, GH, CA 72-4 and CA 19-8, wherein dysregulation of the at least two, three, four or five markers indicates high specificity and sensitivity for a diagnosis of ovarian cancer.

In still a further embodiment, a method is provided for early diagnosis of ovarian cancer in a patient comprising determining the levels of at least one marker from each of the following functional groups: cancer antigens such as CA-125, CEA, CA 72-4, CA 19-9 and CA 15-3; cytokines such as MIF, G-CSF, IL-8, MIP-1b, MCP-1, IL-2R, IL-6, TNF-α, IP-10, MIP-1a and TNFR I; hormones such as FSH, resistin, GH, LH, ACTH, TSH, SMR, mesothelin (IgY), adiponectin, leptin, kallikrein 8, kallikrein 10, MPO, prolactin, HE4 and AFP; growth/angiogenic factors such as EGFR, HGF, ErbB2, IGFPB-1, VEGF and NGF; metastasis-related molecules such as MMP-2, MMP-3, PAI-I (active), sE-selectin, sV-CAM, cytokeratin, sI-CAM and tPAI 1; and apoptosis-related molecules such as sFASL, sFAS, Fas and FAS L, wherein dysregulation of the at least one marker from each of the functional groups indicates high specificity and sensitivity for a diagnosis of ovarian cancer.

In still another embodiment, a method is provided for early diagnosis of ovarian cancer in a patient comprising determining the levels of markers in a blood marker panel comprising at least two or at least five of EGF, G-CSF, IL-6, IL-8, CA-125, VEGF, MCP-1, cytokeratin 19, EGFR, CEA, kallikrein-8, M-CSF, FAS L, ErbB2 and Her2/neu, wherein dysregulation of the at least two or all five markers indicates high specificity and sensitivity for a diagnosis of ovarian cancer.

In still a further embodiment, a method is provided for early diagnosis of ovarian cancer in a patient comprising determining the levels of markers in a blood marker panel comprising at least two or at least ten of CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI 1, CA 15-3, TNF-RI, FAS L, VEGF and NGF, wherein dysregulation of the at least two or at least ten markers compared to a control sample comprised of patients with benign pelvic tumors indicates high specificity and sensitivity for a diagnosis of ovarian cancer.

In still another embodiment, an array is provided comprising binding reagent types specific to any two or more of EGF, G-CSF, IL-6, IL-8, CA-125, VEGF, MCP-1, anti-c-myc, anti-p53, anti-CEA, anti-CA 15-3, anti-MUC-1, anti-survivin, anti-bHCG, anti-osteopontin, anti-PDGF, cytokeratin 19, CEA, kallikrein-8, M-CSF, EGFR and Her2/neu, wherein each binding reagent type is attached independently to one or more discrete locations on one or more surfaces of one or more substrates. The substrates may be beads comprising an identifiable marker, wherein each binding reagent type is attached to a bead comprising a different identifiable marker than beads to which a different binding reagent is attached. The identifiable marker may comprise a fluorescent compound or a quantum dot.

In still a further embodiment, an array is provided comprised of binding reagent types specific to a panel of eight blood markers comprised of CA-125, CA-19-9, EGFR, eotaxin, G-CSF, IL-2R (optionally substituted with prolactin), sV-CAM and MIF, in which each binding reagent type is attached independently to one or more discrete locations on one or more surfaces of one or more substrates. The substrates may be beads comprising an identifiable marker, wherein each binding reagent type is attached to a bead comprising a different identifiable marker than beads to which a different binding reagent is attached. The identifiable marker may comprise a fluorescent compound or a quantum dot.

In still another embodiment, a method is provided to predict the onset of ovarian cancer in a patient, comprised of determining the change in concentration at two or more time points of CA-125, CA-19-9, EGFR, eotaxin, G-CSF, IL-2R (optionally substituted with prolactin), sV-CAM and MIF in a patient's blood, wherein an increase in the serum levels of CA-125, CA-19-9, IL-2R, MIF and prolactin in the patent's blood between the two time points and a decrease in the serum levels of EGFR, eotaxin and sV-CAM in the patient's blood between the two time points are predictive of the onset of ovarian cancer.

In still a further embodiment, a method is provided for comparing the serum levels of the markers set forth herein in a blood marker panel with levels of the same markers in one or more control samples by applying a statistical method such as linear regression analysis, classification tree analysis and heuristic na\dot{i}ve Bayes analysis.

To classify patients as either normal controls or ovarian cancer cases, a variety of different classification methods can be implemented including logistic regression, classification trees; and neural networks. All analyses can be conducted using S-Plus statistical software. Each of the classification methods, which are described in further detail in the subsequent paragraphs, is implemented using 10-fold cross-validation (Efron and Tibshirani, 2000) to minimize bias of resulting classification rates. Classification accuracy is judged via the overall classification rate, sensitivity, specificity, and the receiver operating characteristic (ROC) curve. The ROC curve plots the sensitivity by 1 specificity across a range of cut-points. In other words, analysis begins by classifying all patients as a case and then the required predicted probability from 0.0 to 1.0 is increased (in 0.01 increments).

In each case, all estimates of classification accuracy (including the ROC curves) are calculated within the framework of 10-fold cross-validation. For each of the classification methods, the number of predictor variables is limited based on a univariate Wilcoxon rank-sum test, which assesses the significance of the difference in ranks between cases and controls for the given marker. The rank-sum test is the non-parametric analog to the two-sample unpaired t-test. In the case of classification trees (which automatically includes a variable selection procedure as described in subsequent paragraphs), classification results are obtained using both the entire set of variables and those that are statistically significant with the Wilcoxon test.

Ten-fold cross-validation is implemented by first randomly partitioning the data into ten subsets. The same ten subsets are utilized for each of the subsequently described classification methods, so that classification results are comparable across different methods. The first nine subsets then are used to fit the model, and the last subset is used to calculate classification rates. The process is repeated ten times with a different subset selected each time for testing and the remaining subsets used for training.

Classification trees first were used to predict cancer status (Brieman et al., 1984). Classification trees are a non-parametric classification method that divide subjects into homogeneous subgroups of decreasing size and assign a probability of the given outcome to each group. More specifically, the methods of the present invention can use a technique called recursive partitioning, which searches the range of each potential predictor or marker and finds the split which best divides the data into cases and controls. The process continues until the outcome is perfectly divided or the data are too sparse (e.g. n<5) for further classification. The proportion of cases in the final resulting subsets (i.e., terminal nodes) is used as the estimated predicted probability for corresponding test set observations. Results of the classification analysis also can be visually displayed using a decision tree to show the specific classification rules.

Logistic regression then is implemented to classify cases from controls. The logistic model is a standard parametric approach for classification of binary outcomes that calculates the predicted probability of an event (ovarian cancer) as the logistic function of the weighted sum of the predictor variables, where the logistic function is defined as f(z)=(1+e^{-z})⁻¹. For the logistic model, the set of predictor variables first is limited to those markers which are identified as statistically significant (p<0.05) from the rank-sum test.

Feed-forward neural networks also are implemented for classification analysis. Neural networks are an inherently non-linear parametric method that are universal approximators and may produce more accurate classification than standard methods such as logistic regression. The network response function can be stated as

$$\hat{y} = f ( {α}_{0} + {\sum }_{j} {α}_{j} f ( {β}_{0 j} + {\sum }_{i} {β}_{ij} {x}_{i} ) ) ,$$

where f again is the logistic function and each

$$f ( {β}_{0 j} + {\sum }_{i} {β}_{ij} {x}_{i} )$$

is referred to as the j^{th} hidden unit. The model therefore is related to the logistic model, except that the logistic function of the weighted sum of separate logistic functions is taken. The model therefore is an inherently non-linear function of the data which implicitly fits interactions and non-linear terms (which can be formally shown via a Taylor's series expansion (Landsittel et al., 2002)).

In a typical study, the number of hidden units can be varied, for example and without limitation, from a minimum of 2 to a maximum of 30 (where classification results appear to stabilize). A weight decay term (of 0.01), which is a penalized likelihood function, also can be incorporated to improve model fit and generalizability. The S-Plus algorithm uses an iterative fitting method based on maximizing the likelihood to calculate the optimal coefficients. The maximum number of iterations can be increased, for example and without limitation, to 1,000 (from the default value of 100).

It is understood that the LO and HI values for each of the blood markers are approximate and are derived statistically. By using other statistical methods to detect the relative levels of each factor and to define the critical values for HI and LO, values slightly above or below, typically within one standard deviation of those approximate values might be considered as statistically significant values for distinguishing the LO or HI state from normal. For this reason, the word “about” is used in connection with the stated values. “Statistical classification methods” are used to identify markers capable of discriminating normal patients and patients with benign tumors with ovarian cancer patients and are used to determine critical blood values for each marker for discriminating such patients. The present invention can use, for example and without limitation, three different statistical methods to identify the discriminating markers. These statistical methods include, without limitation: 1) linear regression; 2) classification tree methods (CART), along with CHAID and QUEST; and 3) statistical machine learning to optimize the unbiased performance of algorithms for predicting the masked class labels. Each of these statistical methods is well known to those of ordinary skill in the field of biostatistics and can be performed as a process in a computer. A large number of software products is available commercially to implement statistical methods, such as, without limitation, S-PLUS®, commercially available from Insightful Corporation of Seattle, Wash.

The term “binding reagent” and like terms refers to any compound, composition or molecule capable of specifically or substantially specifically (that is with limited cross-reactivity) binding another compound or molecule, which, in the case of immune-recognition, is an epitope. A “binding reagent type” is a binding reagent or population thereof having a single specificity. The binding reagents typically are antibodies, preferably monoclonal antibodies, or derivatives or analogs thereof, but also include, for example and without limitation: Fv fragments; single chain Fv (scFv) fragments; Fab′ fragments; F(ab′)2 fragments; humanized antibodies and antibody fragments; camelized antibodies and antibody fragments; and multivalent versions of the foregoing. Multivalent binding reagents also may be used as appropriate, which include, without limitation: monospecific or bispecific antibodies, such as disulfide stabilized Fv fragments; scFv tandems ((scFv)2 fragments); or diabodies, tribodies or tetrabodies, which typically are covalently linked or otherwise stabilized (i.e., leucine zipper or helix stabilized) scFv fragments. “Binding reagents” also include aptamers, as are described in the art.

Methods of making antigen-specific binding reagents, including antibodies and their derivatives and analogs and aptamers, are well known in the art. Polyclonal antibodies can be generated by immunization of an animal. Monoclonal antibodies can be prepared according to standard (hybridoma) methodology. Antibody derivatives and analogs, including humanized antibodies can be prepared recombinantly by isolating a DNA fragment from DNA encoding a monoclonal antibody and subcloning the appropriate V regions into an appropriate expression vector according to standard methods. Phage display and aptamer technology is described in the literature and permit in vitro clonal amplification of antigen-specific binding reagents with very high affinity low cross-reactivity. Phage display reagents and systems are available commercially, and include the Recombinant Phage Antibody System (RPAS), commercially available from Amersham Pharmacia Biotech, Inc. of Piscataway, N.J. and the pSKAN Phagemid Display System, commercially available from MoBiTec, LLC of Marco Island, Fla. Aptamer technology is described, for example and without limitation, in U.S. Pat. Nos. 5,270,163, 5,475,096, 5,840,867 and 6,544,776.

The Luminex LabMAP bead-type immunoassay described below is an example of a sandwich assay. The term “sandwich assay” refers to an immunoassay where the antigen is sandwiched between two binding reagents, which typically are antibodies; the first binding reagent/antibody being attached to a surface and the second binding reagent/antibody comprising a detectable group. Examples of detectable groups include, for example and without limitation, fluorochromes; enzymes; or epitopes for binding a second binding reagent, i.e., when the second binding reagent/antibody is a mouse antibody, which is detected by a fluorescently-labeled anti-mouse antibody, for example, an antigen or a member of a binding pair, such as biotin. The surface may be a planar surface, such as in the case of a typical grid-type array, for example and without limitation, 96-well plates and planar microarrays, as described herein, or a non-planar surface, as with coated bead array technologies, where each “species” of bead is labeled with, for example, a fluorochrome, such as the Luminex technology described herein and in U.S. Pat. Nos. 6,599,331, 6,592,822 and 6,268,222, or quantum dot technology, for example, as described in U.S. Pat. No. 6,306,610.

The LabMAP system incorporates polystyrene microspheres that are dyed internally with two spectrally distinct fluorochromes. Using precise ratios of these fluorochromes, an array is created consisting of 100 different microsphere sets with specific spectral addresses. Each microsphere set can possess a different reactant on its surface. Because microsphere sets can be distinguished by their spectral addresses, they can be combined, allowing up to 100 different analytes to be measured simultaneously in a single reaction vessel. A third fluorochrome coupled to a reporter molecule quantifies the biomolecular interaction that has occurred at the microsphere surface. Microspheres are interrogated individually in a rapidly flowing fluid stream as they pass by two separate lasers in the Luminex analyzer. High-speed digital signal processing classifies the microsphere based on its spectral address and quantifies the reaction on the surface in a few seconds per sample.

For the assays described herein, the bead-type immunoassays are preferable for a number of reasons. As compared to ELISAs, costs and throughput are far superior. As compared to typical planar antibody microarray technology (for example, in the nature of the BD Clontech Antibody arrays, commercially available form BD Biosciences Clontech of Palo Alto, Calif.), the beads are far superior for quantification purposes because the bead technology does not require pre-processing or titering of the plasma or serum sample, with its inherent difficulties in reproducibility, cost and technician time. For this reason, although other immunoassays, such as ELISA, RIA and antibody microarray technologies, are capable of use in the context of the present invention, they are not preferred. As used herein, “immunoassays” refer to immune assays, typically, but not exclusively, sandwich assays, capable of detecting and quantifying the eight blood markers simultaneously, namely CA-125, CA-19-9, EGFR, eotaxin, G-CSF, IL-2R, sV-CAM, MIF and optionally prolactin substituted for IL-2R.

Data generated from an assay to determine blood levels of these markers can be used to diagnose ovarian cancer in the patient. As shown herein, if serum levels of markers in a blood marker panel of CA-125, CA-19-9, IL-2R, MIF and optionally prolactin substituted for IL-2R are significantly increased, and serum levels of eotaxin and MCP-1 are significantly decreased, compared to healthy matched controls or patients with benign ovarian tumors, then there is a very high likelihood that the patient has ovarian cancer.

In the context of the present disclosure, “blood” includes any blood fraction, for example, serum, which can be analyzed according to the methods described herein. Serum is a standard blood fraction that can be tested, and is tested in the Examples below. By measuring blood levels of a particular marker, it is meant that any appropriate blood fraction can be tested to determine blood levels and that data can be reported as a value present in that fraction. As a non-limiting example, the blood levels of a marker can be presented as 50 pg/mL serum.

As described above, methods for diagnosing ovarian cancer by determining levels of specifically identified blood markers are provided. Also provided are methods of detecting preclinical ovarian cancer, comprising determining the presence and/or velocity of specifically identified markers in a patient's blood. By velocity, it is meant changes in the concentration of the marker in a patient's blood over time, for example and without limitation, between two time points.

The present invention is more particularly described in the following non-limiting example, which is intended to be illustrative only, as numerous modifications and variations therein will be apparent to those skilled in the art.

### EXAMPLE 1

### Multiplexed Serum Assay for Early Detection of Ovarian Cancer

1. Patient Population, Materials and Methods

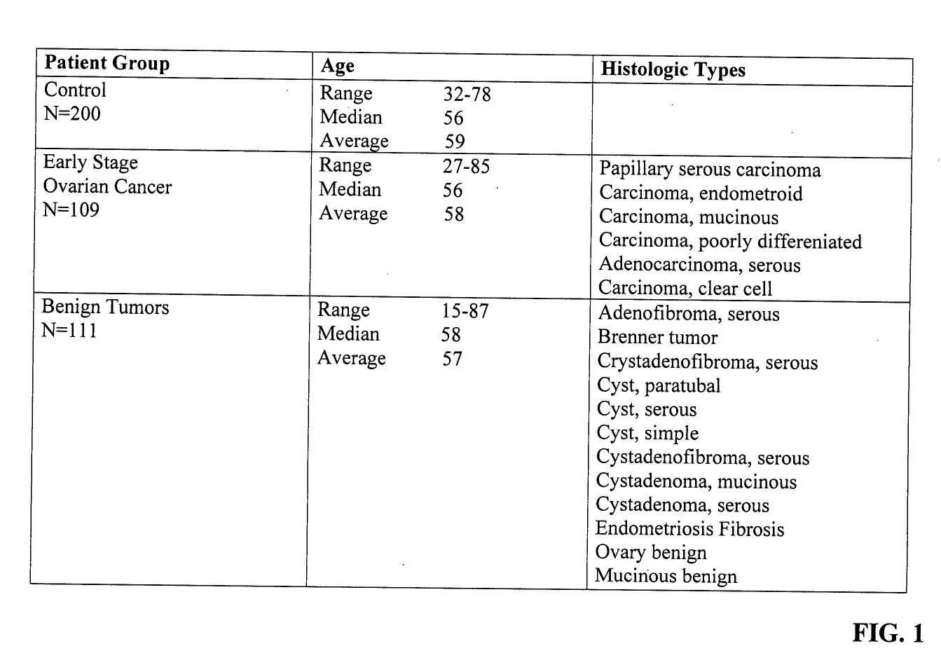

Patient Populations. Serum samples from 109 patients diagnosed with stage (I-II) ovarian cancer, 111 patients with benign pelvic masses and 200 age- and sex-matched healthy controls were tested. Serum samples from patients with documented ovarian cancer were collected under an IRB approved protocol. Serum samples from patients with benign pelvic masses were obtained from the University of Pittsburgh, Division of Gastroenterology under a separate IRB approved protocol. Healthy controls were recruited as a part of ongoing translational research studies within the UPCI Early Detection Research Network/Biomarker Detection Laboratory (EDRN/BDL). The breakdown of the three populations with respect to age and histologic types of ovarian cancer and benign tumors is shown in FIG. 1. Written informed consent was obtained from each subject before sample collection. All samples from the three populations were drawn, processed, and stored under stringent conditions as described below.

Peripheral blood samples were collected following informed consent using standard venipuncture techniques into sterile 10 ml BD Vacutainer™ glass serum (red top) tubes (BD, Franklin Lakes, N.J.) and left to stand undisturbed for 30 minutes at room temperature. The tubes then were spun at room temperature at 20×100 rpm for 10 minutes in a Sorvall benchtop centrifuge. The serum fraction then was carefully collected by pipetting into a pre-chilled tube on ice and mixed to ensure homogeneity of the serum sample. The serum then was divided into 1.0 ml aliquots in pre-chilled 1.8 ml Cryovial tubes on ice. The aliquots then were stored at −80° C. or below. Processing time from phlebotomy to freezing at −80° C. was within one hour. Immediately prior to analysis, serum aliquots were thawed on ice with intermittent agitation to avoid the formation of precipitate. No more than two freeze-thaw cycles were allowed for each sample.

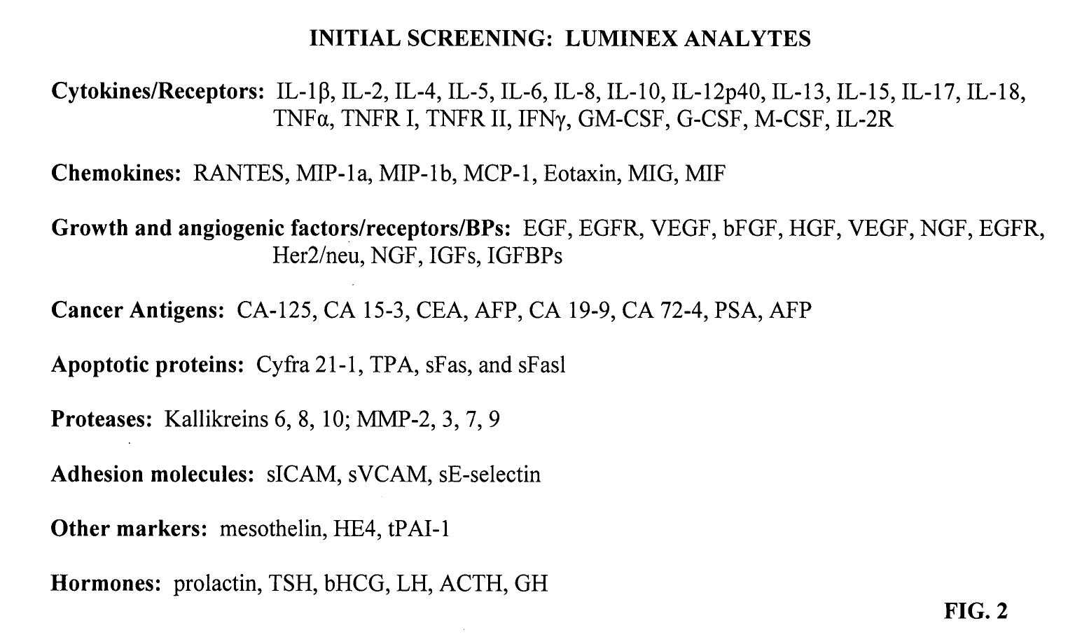

Initial Screening: Luminex Analytes. An initial screening of at least 46 analytes, which included cytokines/receptors; chemokines; growth and angiogenic factors/receptors; cancer antigens; apoptotic proteins; proteases; adhesion molecules; hormones and other markers, using the LabMAP assay developed in our laboratory (described previously in Gorelik, E. et al., Multiplexed Immunobead-Based Cytokine Profiling for Early Detection of Ovarian Cancer, Cancer Epidemiology Biomarkers and Prevention, In Press, 2004), was performed on each serum sample using kits purchased from BioSource International (Camarillo, Calif.). (FIG. 2). The LabMAP™ serum assays were performed in 96-well microplate format as described above.

For each LabMAP™ assay, a proprietary combination of two specific antibodies, monoclonal capture and polyclonal detection, was utilized. The detection antibody was biotinylated using the EZ-Link Sulfo-NHS-Biotinylation Kit (Pierce, Rockford, Ill.) according to the manufacturer's protocol. The capture antibody was covalently coupled to individually spectrally addressed carboxylated polystyrene microspheres purchased from Luminex Corp. The minimum detection level for each analyte was <3.3 pg/ml. Inter-assay variability, expressed as a coefficient of variation (CV), was calculated based on the average for ten patient samples and standards that were measured in four separate assays. The inter-assay variability within the replicates presented as an average CV was 8.7-11.2% (data not shown). Intra-assay variability was evaluated by testing quadruplicates of each standard and ten samples measured three times. The CVs of these samples were between 6.9 and 9.8% (data not shown). In addition, the percent recovery from serum was 96-98% and correlations with standard ELISAs (Calbiotech, Spring Valley, Calif.) were 92-94%.

Statistical Analysis of Data. Descriptive statistics and graphical displays (i.e., dot plots) were prepared to show the distribution of the serum level of each marker for each disease state. The Wilcoxon rank-sum test was used to evaluate the significance of differences in marker expression between each disease state. Spearman's (nonparametric) rank correlation also was calculated to quantify the relationships between each pair of markers.

Discrimination of ovarian cancer status was accomplished using classification trees (CART) (Brieman, F. J et al., Classification and Regression Trees, 1984, Monterey: Wadsworth and Brooks/Cole) implemented through S-Plus statistical software (Venables, W. et al., Modern applied statistics with S-plus, 1997, New York: Springer-Verlag), which classifies subjects into homogeneous subgroups of decreasing size and assigns a probability of the given outcome to each group. These groups then are drawn on a decision tree to show the specific rules used for classification. Comparisons were repeated for ovarian cancer versus normal controls, and ovarian cancer versus benign pelvic masses.

For comparisons of cancer versus normal controls, and cancer versus benign pelvic masses, subjects with a predicted probability greater than or equal to 0.5 (using the classification tree model) were classified as cancerous, and all others (predicted probability less than 0.5) as non-cancerous (i.e., controls or benign pelvic masses). To appropriately evaluate classification results, 10-fold cross-validation (Tibshirani, R. et al., Statist. Applic. Genet. Mol. Biol., 1, 2002; Efron, R. et al., J. Amer. Statist. Assoc. 96:1151-1160, 2001), also was implemented to provide a more unbiased measure of classification accuracy (as opposed to simply evaluating classification results on the same data used to fit the model, which is known to be optimistically biased and prone to overfitting). Sensitivity, specificity, and the overall classification rate were calculated to quantify classification accuracy. The classification trees presented for each comparison represent the model fit to the entire data set. The ROC curves utilized 10-fold cross-validation to produce all classification results.

2. Results

LabMAP™-Based Analysis of Serum Concentrations of Blood Markers in Ovarian Cancer Patients. Concentrations of at least 46 different serum markers belonging to different biological functional groups were evaluated in a multiplexed assay using LabMAP™ technology in serum samples of patients from three clinical groups: ovarian cancer patients, patients with benign pelvic masses, and control healthy subjects who were matched to disease groups by age, sex and smoking status.

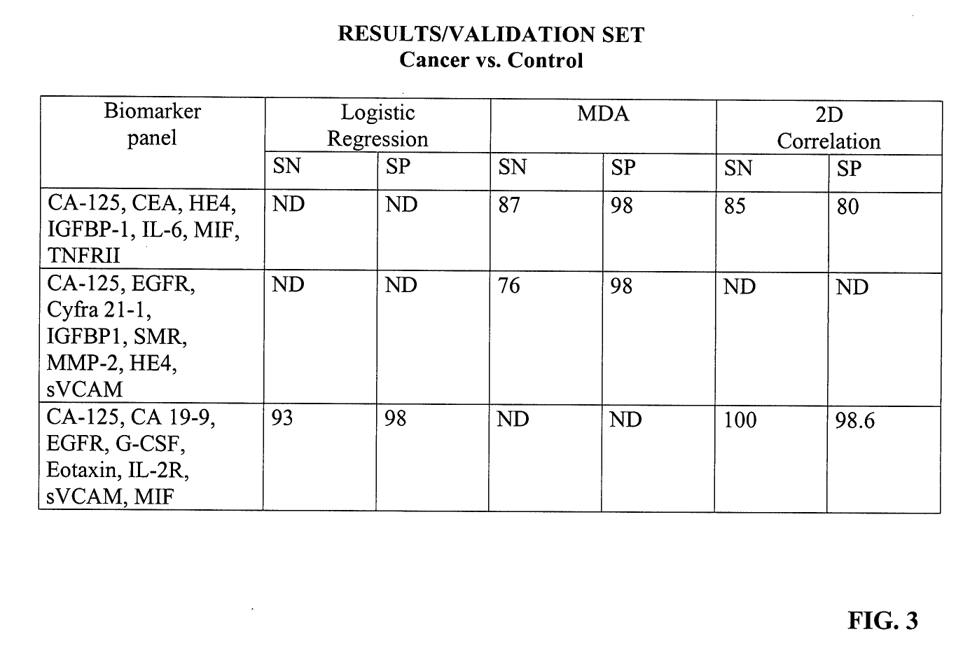

Ovarian Cancer vs. Controls. Multiplexed assay of at least 46 serum markers revealed a group of eight serum markers whose concentrations were significantly different in patients with ovarian cancer as compared to healthy controls. Specifically, serum concentrations of CA-125, CA-19-9, IL-2R (optionally substituted with prolactin; data not shown) and MIF were found to be significantly higher in ovarian cancer patients as compared to controls (FIG. 3). Concentrations of EGFR, eotaxin and sV-CAM were found to be significantly lower in ovarian cancer patients as compared to controls (FIG. 3).

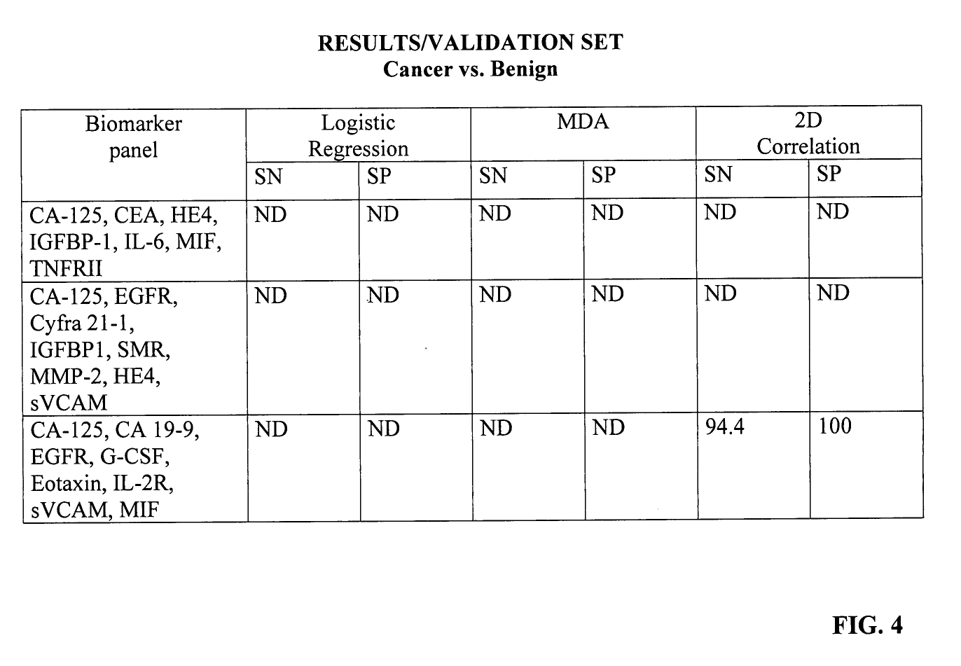

Ovarian Cancer vs. Benign Pelvic masses. Serum cytokine concentrations in patients with ovarian cancer were measured and compared to those patients with benign pelvic masses. This comparison identified the same eight markers demonstrating significant differences in serum concentrations between these two clinical groups. Specifically, serum concentrations of CA-125, CA-19-9, IL-2R (optionally substituted with prolactin; data not shown) and MIF were found to be significantly higher in ovarian cancer patients as compared to controls (FIG. 4). Concentrations of EGFR, eotaxin and sV-CAM were found to be significantly lower in ovarian cancer patients as compared to controls (FIG. 4).

3. Discussion

Multiplexed LabMAP™ technology was utilized for analysis of at least 46 blood markers in sera of patients with ovarian cancer in comparison with patients with benign pelvic tumors and matched healthy controls. To our knowledge, this is the largest panel of blood markers to be examined simultaneously in ovarian cancer. The sensitivity of the LabMAP™ assays was comparable to ELISA and RIA [R. T. Carson, R. T. et al., Immunol. Methods, 227:41-52, 1999).

Eight blood markers were identified that showed an association with ovarian cancer versus healthy matched controls and patients with benign pelvic/ovarian masses: CA-125, CA-19-9, EGFR, eotaxin, G-CSF, IL-2R (optionally substituted with prolactin), sV-CAM and MIF. Two patterns of changes were observed: the serum concentrations of CA-125, CA-19-9, IL-2R, MIF and prolactin were higher; whereas concentrations of EGFR, eotaxin and sV-CAM were decreased in patients with ovarian cancer in comparison to the controls.

Statistical analysis demonstrated that although correlation of each of the identified markers with ovarian cancer was modest when evaluated alone, a combined biomarker panel showed very strong association with malignant disease. Combinations of several serum markers as measured by LabMAP™ technology provided a sensitivity of 100% at a specificity of 98.6% for comparison of ovarian cancer with healthy controls, and a sensitivity of 94.4% at a specificity of 100% for comparison of ovarian cancer with benign pelvic masses. This panel has demonstrated higher performance than any published single ovarian cancer-associated marker (Hayakawa, T. et al., Int. J. Pancreatol., 25: 23-9, 1999; Carpelan-Holmstrom, M. et al., Anticancer Res., 22: 2311-6, 2002), or marker combination (Mor, G. et al., PNAS, 102:7677-7682, 2005; Hayakawa, T. et al., Int. J. Pancreatol., 25: 23-9, 1999; Carpelan-Holmstrom, M. et al., Anticancer Res., 22: 2311-6, 2002).

The ability to discriminate between patients with benign tumors of the ovaries and malignancy is of significant clinical importance. Current diagnostic modalities are inadequate in that ovarian cancer seldom is diagnosed early in the disease. These results demonstrate that the blood marker panel can serve as an extremely efficient discriminator between and ovarian cancer and benign pelvic masses.

It will be appreciated by those skilled in the art that changes could be made to the embodiments described above without departing from the broad inventive concept thereof. it is understood, therefore, that this invention is not limited to the particular embodiments disclosed, but it is intended to cover modifications that are within the spirit and scope of the invention, as defined by the appended claims.

The invention claimed is:

1. A method of diagnosing ovarian cancer in a patient, comprising determining the levels of at least four markers in the blood of a patient, wherein at least two different markers are selected from the group consisting of CA-125, prolactin, HE4, sV-CAM and TSH, and wherein a third marker and a fourth marker are selected from the group consisting of CA-125, prolactin, HE4, sV-CAM(16), TSH, cytokeratin, sI-CAM, IGFBP-1, eotaxin and FSH, and further wherein each of said third marker and said fourth marker is different from the other and different from either of said at least two markers, wherein dysregulation of said at least four markers indicates high specificity and sensitivity for a diagnosis of ovarian cancer.

2. A method of diagnosing ovarian cancer in a patient, comprising determining the levels of at least six markers in the blood of a patient, wherein at least three different markers are selected from the group consisting of CA-125, prolactin, HE4, sV-CAM and TSH, and wherein a fourth marker, a fifth marker and a sixth marker are selected from the group consisting of CA-125, prolactin, HE4, sV-CAM, TSH, cytokeratin, sI-CAM, IGFBP-1, eotaxin and FSH, and further wherein each of said fourth marker and said fifth marker and said sixth marker is different from the other and is different from any of said at least three markers, wherein dysregulation of said at least six markers indicates high specificity and sensitivity for a diagnosis of ovarian cancer.

3. A method of diagnosing ovarian cancer in a patient, comprising determining the levels of at least eight markers in the blood of a patient, wherein at least four different markers are selected from the group consisting of CA-125, prolactin, HE4, sV-CAM and TSH, and wherein a fifth marker, a sixth marker, a seventh marker and an eighth marker are selected from the group consisting of CA-125, prolactin, HE4, sV-CAM, TSH, cytokeratin, sI-CAM, IGFBP-1, eotaxin and FSH, and further wherein each of said fifth marker, said sixth marker, said seventh marker and said eighth marker is different from the other and is different from any of said at least four markers, wherein dysregulation of said at least eight markers indicates high specificity and sensitivity for a diagnosis of ovarian cancer.

4. A method of diagnosing ovarian cancer in a patient, comprising determining levels of eight markers in a blood marker panel, comprising CA-125, CA-19-9, EGFR, eotaxin, G-CSF, IL-2R, sV-CAM and MIF, wherein IL-2R optionally is substituted with prolactin.

5. The method according to claim 4, wherein the presence of the following conditions indicates the presence of ovarian cancer in the patient: CA-125_{HI}, CA-19-9_{HI}, EGFR_{LO}, eotaxin_{LO}, IL-2R_{HI}, SV-CAM_{LO}, MIF_{HI} and prolactin_{HI}.

6. The method according to claim 4, further comprising comparing the levels of the eight blood markers in the patient's blood with levels of the same markers in a control sample comprised of healthy patients by applying a statistical method selected from the group consisting of linear regression analysis, classification tree analysis and heuristic naive Bayes analysis.

7. The method according to claim 4, further comprising comparing the levels of the eight blood markers in the blood of patients with ovarian cancer with levels of the same markers in a control sample comprised of patients with benign pelvic tumors by applying a statistical method selected from the group consisting of linear regression analysis, classification tree analysis and heuristic naive Bayes analysis.

8. The method according to claim 6, wherein the statistical method is performed by a computer process.

9. The method according to claim 6, wherein the statistical method is a classification tree analysis.

10. The method according to claim 6, wherein the blood marker panel in which the levels of blood markers of patients afflicted with ovarian cancer is compared fo the control sample of healthy women generates a sensitivity of at least 90% and a specificity of at least 90%.

11. The method according to claim 6, wherein the blood marker panel in which the levels of blood markers of patients afflicted with ovarian cancer is compared to the control sample of healthy women generates a sensitivity of at least 98% and a specificity of at least 98%.

12. The method according to claim 7, wherein the blood marker panel in which the levels of blood markers of patients afflicted with ovarian cancer are compared to the control sample of patients diagnosed with benign pelvic tumors generates a sensitivity of at least 90% and a specificity of at least 90%.

13. The method according to claim 7, wherein the blood marker panel in which the levels of blood markers of patients afflicted with ovarian cancer are compared to the control sample of patients diagnosed with benign pelvic tumors generates a sensitivity of at least 92% and a specificity of 98%.

14. The method according to claim 4, wherein the blood sample is a serum sample.

15. The method according to claim 4, wherein the levels of markers in the blood marker panel are determined by performing an immunoassay.

16. The method according to claim 15, wherein the immunoassay utilizes an array comprising binding reagent types specific to CA-125, CA-19-9, EGFR, eotaxin, G-CSF, IL-2R, sV-CAM, MIF and prolactin, and wherein each binding reagent type is attached independently to one or more discrete locations on one or more surfaces of one or more substrates.

17. The method according to claim 16, wherein the substrates are beads comprising an identifiable marker, and wherein each binding reagent type is attached to a bead comprising a different identifiable marker than beads to which a different binding reagent type is attached.

18. The method according to claim 17, wherein the identifiable marker comprises a fluorescent compound.

19. The method according to claim 17, wherein the identifiable marker comprises a quantum dot.

20. An array comprising binding reagent types specific to CA-125, CA-19-9, EGFR, eotaxin, G-CSF, IL-2R, sV-CAM, MIF and prolactin, wherein each binding reagent type is attached independently to one or more discrete locations on one or more surfaces of one or more substrates.

21. The array of claim 20, wherein the substrates are beads comprising an identifiable marker, and wherein each binding reagent type is attached to a bead comprising a different identifiable marker than beads to which a different binding reagent type is attached.

22. The array according to claim 20, wherein the identifiable marker comprises a fluorescent compound.

23. The array according to claim 20, wherein the identifiable marker comprises a quantum dot.

24. A method of predicting onset of ovarian cancer in a patient, comprising determining the change in blood levels at two or more time points of CA-1 25, CA-1 9-9, EGFR, eotaxin, G-CSF, IL-2R, sV-CAM, MIF and optionally IL-2R substituted with prolactin in the patient's blood, wherein an increase in the serum levels of CA-125, CA-19-9, IL-2R, MIF and prolactin in the patent's blood between the two time points and a decrease in the serum levels of EGFR, eotaxin and sV-CAM in the patient's blood between the two time points are predictive for the onset of ovarian cancer in the patient.

25. A method of diagnosing ovarian cancer in a patient, comprising determining the levels of markers in a blood marker panel comprising at least two of CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least two markers indicates high specificity and sensitivity for a diagnosis of ovarian cancer.

26. The method of claim 25, wherein the panel comprises at least three of CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least three markers indicates high specificity and sensitivity for a diagnosis of ovarian cancer.

27. The method of claim 25, wherein the panel comprises at least four of CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least four markers indicates high specificity and sensitivity for a diagnosis of ovarian cancer.

28. The method of claim 25, wherein the panel comprises at least five of CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein 8, leptin, kallikrein 10,MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI 1, CA 15-3, TNF-RI, FAS L, VEGF and NGF, wherein dysregulation of said at least five markers indicates high specificity and sensitivity for a diagnosis of ovarian cancer.

29. The method of claim 25, wherein the panel comprises at least six of CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least six markers indicates high specificity and sensitivity for a diagnosis of ovarian cancer.

30. The method of claim 25, wherein the panel comprises at least seven of CA-125, prolactin, HE4, sV-CAM, TSH, cytokeratin, sI-CAM, IGFPB-1, eotaxin, FSH, MMP-2, MIF, sFASL, CEA, mesothelin (IgY), EGFR, CA 72-4, GH, CA 19-9, IL-8, MIP-1b, LH, MCP-1, MMP-3, MPO, sE-selectin, IL-6, TNF-a, ErbB2, AFP, IP-10, ACTH, HGF, IL-2R, SMR, kallikrein-10, MIP-1a, Fas, tPAI-1, CA 15-3, TNF-RI, adiponectin, PAI-I (active), sFAS, kallikrein-8, leptin, VEGF, resistin, G-CSF, NGF and FAS L, wherein dysregulation of said at least seven markers indicates high specificity and sensitivity for a diagnosis of ovarian cancer.

31. The method of claim 25, wherein the panel comprises at least eight of CA-125, eotaxin, FSH, MMP-2, MIF, sFASL, CEA, resistin, G-CSF, mesothelin (Igy), EGFR, CA 72-4, GH, CA 19-9, IL-8, MIP-lb, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least eight markers indicates high specificity and sensitivity for a diagnosis of ovarian cancer.

32. The method of claim 25, wherein the panel comprises at least nine of CA-125, eotaxin, FSH, MMP-2, MIF, sFASL, CEA, resistin, G-CSF, mesothelin (Igy), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least nine markers indicates high specificity and sensitivity for a diagnosis of ovarian cancer.

33. The method of claim 25, wherein the panel comprises at least ten of CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least ten markers indicates high specificity and sensitivity for a diagnosis of ovarian cancer.

34. The method of claim 25, wherein the panel comprises at least eleven of CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least eleven markers indicates high specificity and sensitivity for a diagnosis of ovarian cancer.

35. The method of claim 25, wherein the panel comprises at least twelve of CA-125, eotaxin, FSH, MMP-2, MIF, sFASL, CEA, resistin, G-CSF, mesothelin (Igy), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least twelve markers indicates high specificity and sensitivity for a diagnosis of ovarian cancer.

36. The method of claim 25, wherein the panel comprises at least thirteen of CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least thirteen markers indicates high specificity and sensitivity for a diagnosis of ovarian cancer.

37. The method of claim 25, wherein the panel comprises at least fourteen of CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least fourteen markers indicates high specificity and sensitivity for a diagnosis of ovarian cancer.

38. The method of claim 25, wherein the panel comprises at least fifteen of CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least fifteen markers indicates high specificity and sensitivity for a diagnosis of ovarian cancer.

39. The method of claim 25, wherein the panel comprises at least sixteen of CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least sixteen markers indicates high specificity and sensitivity for a diagnosis of ovarian cancer.

40. The method of claim 25, wherein the panel comprises at least seventeen of CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least seventeen markers indicates high specificity and sensitivity for a diagnosis of ovarian cancer.

41. The method of claim 25, wherein the panel comprises at least eighteen of CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least eighteen markers indicates high specificity and sensitivity for a diagnosis of ovarian cancer.

42. The method of claim 25, wherein the panel comprises at least nineteen of CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least nineteen markers indicates high specificity and sensitivity for a diagnosis of ovarian cancer.

43. The method of claim 25, wherein the panel comprises at least twenty of CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least twenty markers indicates high specificity and sensitivity for a diagnosis of ovarian cancer.

44. The method of claim 25, wherein the panel comprises at least twenty-one of CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least twenty-one markers indicates high specificity and sensitivity for a diagnosis of ovarian cancer.

45. The method of claim 25, wherein the panel comprises at least twenty-two of CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least twenty-two markers indicates high specificity and sensitivity for a diagnosis of ovarian cancer.

46. The method of claim 25, wherein the panel comprises at least twenty-three of CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least twenty-three markers indicates high specificity and sensitivity for a diagnosis of ovarian cancer.

47. The method of claim 25, wherein the panel comprises at least twenty-four of CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least twenty-four markers indicates high specificity and sensitivity for a diagnosis of ovarian cancer.

48. The method of claim 25, wherein the panel comprises at least twenty-five of CA-125, eotaxin, FSH, MMP-2, MIF, sFASL, CEA, resistin, G-CSF, mesothelin (Igy), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least twenty-five markers indicates high specificity and sensitivity for a diagnosis of ovarian cancer.

49. A method of diagnosing ovarian cancer in a patient, comprising determining the levels of at least four markers in the blood of a patient, wherein at least one marker is selected from the group consisting of HE4 and eotaxin and wherein other markers are selected from the group consisting of CA-125, prolactin, HE4, sV-CAM, TSH, cytokeratin, sI-CAM, IGFBP-1 and FSH, and further wherein each of said other markers is different from the other and different from either of said at least one marker, wherein dysregulation of said at least four markers indicates high specificity and sensitivity for a diagnosis of ovarian cancer.

50. A method of diagnosing ovarian cancer in a patient, comprising determining the level of one marker selected from the group consisting of TSH, IGFBPI, LH, FSH, sV-CAM, MMP-2, EGFR, ErbB2, GH, CA 72-4 and CA 19-8 in the blood of a patient, wherein dysregulation of said one marker indicates high specificity and sensitivity for a diagnosis of ovarian cancer.

51. A method of diagnosing ovarian cancer in a patient, comprising determining the levels of markers in a blood marker panel comprising at least two of TSH, IGFBPI, LH, FSH, sV-CAM, MMP-2, EGFR, ErbB2, GH, CA 72-4 and CA 19-8, wherein dysregulation of said at least two markers indicates high specificity and sensitivity for a diagnosis of ovarian cancer.

52. The method of claim 51, wherein the panel comprises at least three of TSH, IGFBPI, LH, FSH, sV-CAM, MMP-2, EGFR, ErbB2, GH, CA 72-4 and CA 19-8, wherein dysregulation of said at least three markers indicates high specificity and sensitivity for a diagnosis of ovarian cancer.

53. The method of claim 51, wherein the panel comprises at least four of TSH, IGFBPI, LH, FSH, sV-CAM, MMP-2, EGFR, ErbB2, GH, CA 72-4 and CA 19-8, wherein dysregulation of said at least four markers indicates high specificity and sensitivity for a diagnosis of ovarian cancer.

54. The method of claim 51, wherein the panel comprises at least five of TSH, IGFBPI, LH, FSH, sV-CAM, MMP-2, EGFR, ErbB2, GH, CA 72-4 and CA 19-8, wherein dysregulation of said at least five markers indicates high specificity and sensitivity for a diagnosis of ovarian cancer.

55. A method of diagnosing ovarian cancer in a patient, comprising determining the levels of at least one marker from each of the following functional groups: cancer antigens, cytokines, hormones, growth/angiogenic factors, metastasis-related molecules and apoptosis-related molecules, wherein dysregulation of said at least one marker from each of the finctional groups indicates high specificity and sensitivity for a diagnosis of ovarian cancer.

56. The method of claim 55, wherein the cancer antigen markers are selected from the group consisting of CA-125, CEA, CA 72-4, CA 19-9 and CA 15-3, wherein the cytokine markers are selected from the group consisting of MIF, G-CSF, IL-8, MIP-1b, MCP-1, IL-2R, IL-6, TNF-α, IP-10, MIP-1a and TNFR I, wherein the hormone markers are selected from the group consisting of FSH, resistin, GH, LH, ACTH, TSH, SMR, mesothelin (IgY), adiponectin, leptin, kallikrein-8, kallikrein-10, MPO, prolactin, HE4 and AFP, wherein the growth/angiogenic factors are selected from the group consisting of EGFR, HGF, ErbB2, IGFPB-1, VEGF and NGF, wherein the metastasis-related molecule markers are selected from the group consisting of MMP-2, MMP-3, PAI-I (active), se-selectin, sV-CAM, cytokeratin, sI-CAM and tPAI-1, and wherein the apoptosis-related molecule markers are selected from the group consisting of sFASL, sFAS, Fas and FAS L.

57. A method of diagnosing ovarian cancer in a patient, comprising determining the levels of markers in a blood marker panel comprising at least two of EGF, G-CSF, IL-6, IL-8, CA-125, VEGF, MCP-1, cytokeratin 19, EGFR, CEA, kallikrein-8, M-CSF, FAS L, ErbB2 and Her2/neu, wherein dysregulation of said at least two markers indicates high specificity and sensitivity for a diagnosis of ovarian cancer.

58. The method of claim 57, wherein the panel comprises at least five of EGF, G-CSF, IL-6, IL-8, CA-125, VEGF, MCP-1, cytokeratin 19, EGFR, CEA, kallikrein-8, M-CSF, FAS L, ErbB2 and Her2/neu, wherein dysregulation of said at least five markers indicates high specificity and sensitivity for a diagnosis of ovarian cancer.

59. A method of diagnosing ovarian cancer in a patient, comprising determining the levels of markers in a blood marker panel comprising at least two of CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein 8, leptin, kallikrein 10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least two markers compared to a control sample comprised of patients with benign pelvic tumors indicates high specificity and sensitivity for a diagnosis of ovarian cancer.

60. The method of claim 59, wherein the panel comprises at least ten of CA-125, eotaxin, FSH, MMP-2, MIF, sFASL, CEA, resistin, G-CSF, mesothelin (IgY), EGFR, CA 72-4, GH, CA 19-9, IL-8, MIP-1b, LH, MCP-1, MMP-3, ACTH, HGF, IL-2R, SMR, adiponectin, PAI-I (active), sFAS, kallikrein-8, leptin, kallikrein-10, MPO, sE-selectin, IL-6, TNF-a, ErbB2, prolactin, HE4, sV-CAM, TSH, cytokeratin, sI-CAM, IGFPB-1, AFP, IP-10, MIP-1a, Fas, tPAI-1, CA 15-3, TNF-RI, FAS L, VEGF and NGF, wherein dysregulation of said at least ten markers compared to a control sample comprised of patients with benign pelvic tumors indicates high specificity and sensitivity for a diagnosis of ovarian cancer.
